(19) United States
(12) Patent Application Publication
(10) Pub. No.: US 2018/0111005 A1
(43) Pub. Date: Apr. 26, 2018

(54) METHOD OF SELECTING BEAM GEOMETRIES

(71) Applicant: KONINKLIJKE PHILIPS N.V., EINDHOVEN (NL)

(72) Inventors: Vaitheeswaran RANGANATHAN, Bangalore (IN); Prashant KUMAR, Bangalore (IN); Gipson JOE ANTO, Kaliyal (NL)

(21) Appl. No.: 15/571,294

(22) PCT Filed: May 11, 2016
(86) PCT No.: PCT/EP2016/060572

§ 371 Date: Nov. 2, 2017

(30) Foreign Application Priority Data
May 28, 2015 (IN) 2677/CHE/2015

(51) Int. Cl. A61N 5/10

(52) U.S. Cl. CPC A61N 5/1031; A61N 2005/1092; A61N 5/1045; A61N 5/1048

(57) ABSTRACT

The invention relates to a method of selecting a set of beam geometries for use in radiation therapy. The method (10) comprises providing (12) a plurality of candidate beam geometries; optimizing (1) a radiation treatment plan with all candidate beam geometries; and computing (14) a cost function value based on all candidate beam geometries. A first beam geometry from the plurality of candidate beam geometries is removed (15) and a first modified cost function value based on the candidate beam geometries without the removed first beam geometry computed (16). The first beam geometry is restored (17). The steps of removing a beam geometry, computing of a modified cost function value and restoring of the removed beam geometry are repeated (R) for all other candidate beam geometries. One or more beam geometries from the plurality of candidate beam geometries based on the modified cost function values are chosen (19).

### FIELD OF THE INVENTION

The invention relates to a method of selecting a set of beam geometries for use in radiation therapy, a system for selecting a set of beam geometries for use in radiation therapy and a computer program for selecting a set of beam geometries for use in radiation therapy, in particular in Intensity Modulated Radiation Therapy (IMRT) and/or Intensity Modulated Particle Therapy (IMPT).

### BACKGROUND OF THE INVENTION

Currently, the beam geometry or beam angle selection process in radiation therapy, in particular in IMRT, is based on the experience of the treatment planners or by a trial-and-error approach. There are many optimization algorithms employed for solving Beam Angle Optimization (BAO) problem, among which Genetic Algorithm (GA) was a commonly used one. Simulated annealing (SA) algorithms have also been used by many investigators. Such exhaustive search algorithms generally explore a large number of candidate solutions to arrive at an optimal beam configuration, which considerably prolongs the entire process of BAO in IMRT. In other works, a score function, introduced to measure the “goodness” of each beamlet at a given beam angle, is used to select some beam angles among a set of candidate beam angles. A hybrid approach to beam angle optimization in IMRT has been described in Bertsimas D, et al. A hybrid approach to beam angle optimization in intensity-modulated radiation therapy. Computers and Operations Research (2012), http://dx.doi.Org/10.1016/j.cor.2012.06.009.

### SUMMARY OF THE INVENTION

It is an object of the present invention to provide a method of selecting a set of beam geometries for use in radiation therapy, a system for selecting a set of beam geometries for use in radiation therapy and a computer program for selecting a set of beam geometries for use in radiation therapy, which result in an improved selection of beam geometries. In particular, it is an object of the present invention to provide a method of selecting a set of beam geometries for use in radiation therapy, a system for selecting a set of beam geometries for use in radiation therapy and a computer program for selecting a set of beam geometries for use in radiation therapy, which result in a fast and accurate selection of beam geometries.

In a first aspect of the invention, a method of selecting a set of beam geometries for use in radiation therapy is presented, wherein the method comprises:
providing a plurality of candidate beam geometries;
optimizing a radiation treatment plan with all candidate beam geometries;
computing a cost function value based on all candidate beam geometries;
removing a first beam geometry from the plurality of candidate beam geometries;
computing a first modified cost function value based on the candidate beam geometries without the removed first beam geometry;
restoring the first beam geometry to the plurality of candidate beam geometries;
repeating the steps of removing a beam geometry, computing of a modified cost function value and restoring of the removed beam geometry for all other candidate beam geometries;
choosing one or more beam geometries from the plurality of candidate beam geometries based on the modified cost function values.

The method of selecting a set of beam geometries for use in radiation therapy according to the invention provides a fast and accurate selection method. According to this method, a plurality of candidate beam geometries is provided. This plurality of candidate beam geometries P represents a pool of beam geometries from which one or more beam geometries are to be selected. The one or more beam geometries selected from the pool or plurality of candidate beam geometries then represents the selected set of beam geometries B that are to be used in a radiation treatment plan. The plurality of candidate beam geometries can be provided, for example, by a user input or can be drawn from templates, which can be stored, for example, in a memory of a system or computer.

For this plurality of candidate beam geometries, a radiation treatment plan is optimized. For example, beam intensities for all candidate beam geometries, including beamlets for each beam geometry, can be optimized. Further, a respective composite cost function value based on all candidate beam geometries, in particular the optimized radiation treatment plan with all candidate beam geometries, is computed. This respective composite cost function value F computed based on all candidate beam geometries can also be referred to as the original cost function value. Preferably, the composite or original cost function value F, which is computed based on all candidate beam geometries, is stored, for example, in a memory of a computer or system.

The cost function value can be computed using a cost function taking into account, for example, beam volumetrics, beam dose and weight to determine the doses delivered to regions of interest, in particular the target volume and structures or organs at risk. In particular, treatment parameters, such as a minimal dose of the target volume, and/or conformity of the dose, and/or tolerance parameters, such as dose-volume costs, and/or dose-volume constraints and/or importance factors, of different regions of interest can be used to define a cost function.

The method further comprises removing a first beam geometry from the plurality of candidate beam geometries. In particular, the dose contribution from a first beam geometry can be removed from the plurality of candidate beam geometries. For example, the first beam geometry can be removed by assigning a beam weight value of zero to this first beam geometry. Preferably, the first beam geometry can also be removed by switching off the first beam geometries monitor unit (MU) in a respective treatment planning system.

Then, a first modified cost function value Fₘ is computed based on the candidate beam geometries without the removed first beam geometry. Preferably, the cost function used to compute the first modified cost function value Fₘ is the same cost function used for computing the composite or original cost function value F based on all candidate beam geometries. The first modified cost function value Fₘ is computed based on the plurality of candidate beam geometries except for the first beam geometry that was previously removed.

The first modified cost function value can be the direct calculated result of the cost function computed based on the plurality of candidate beam geometries without the first removed beam geometry. The first modified cost function value can also be a change of the cost function value computed based on the candidate beam geometries without the removed first beam geometry compared to the original cost function value computed based on all candidate beam geometries or it can be a value derived from the direct calculated result of the cost function or the change of the cost function, like the square, for example.

Typically, when the first beam geometry, or its dose contribution, is removed, the cost function value will probably increase, i.e. a modified cost function value will be larger than the original cost function value (Fₘ>F) in case the removed first beam geometry had usefully contributed to the treatment plan. Typically, if the removed beam geometry is more optimal, the increase in the cost function value will be relatively higher. Likewise, if the removed beam geometry is less optimal, the increase in the cost function value will be relatively lower. In some instances, especially when the number of candidate beam geometries is very large, it is also possible that the cost function value may slightly reduce when a beam geometry is removed, i.e. a modified cost function value will be smaller than the original cost function value (Fₘ<F) due to the presence of local minima.

The first modified cost function value preferably is stored after its computation, for example, in a memory of a system or computer. Further preferably, after a modified cost function value is computed and preferably stored, the original cost function value with all candidate beam geometries is stored back. This can be performed, for example, before or after the previously removed first beam geometry is restored.

After the modified cost function value is computed and preferably stored, the previously removed first beam geometry is restored to the plurality of candidate beam geometries. In particular, the dose contribution from the first beam geometry is restored. For example, the first beam geometry can be restored by assigning a beam weight value not equal to zero or different from zero, in particular larger than zero. Preferably, the beam weight value assigned to the first beam geometry previous to its removal is used for restoring this first beam geometry. The first beam geometry can also be restored by switching on the first beam geometries monitor unit (MU), for example in a treatment planning system.

In the following, these three steps of removing a beam geometry from the plurality of candidate beam geometries, computing a modified cost function value based on the candidate beam geometries without the removed beam geometry and restoring the previously removed beam geometry to the plurality of candidate beam geometries are then repeated for all other candidate beam geometries in the pool of candidate beam geometries. In other words, a modified cost function value based on the candidate beam geometries without a removed beam geometry is computed for all candidate beam geometries provided in the beginning of the method. Preferably, all modified cost function values are stored, for example, in the memory of a system or computer.

Further preferably, the method is implemented in a treatment planning system for clinical application. In particular, it is preferred to use an assignment of a beam weight in a treatment planning system to allow for switching off or switching on the monitor unit (MU) of a beam geometry so that this beam geometry can be technically removed or restored very easily without the need to invalidate a previously optimized value, like a computed dose or an optimized fluence map.

Based on the modified cost function values, one or more beam geometries from the plurality of candidate beam geometries are chosen. This set of one or more chosen or selected beam geometries then can be used in a treatment plan.

Preferably, those beam geometries are chosen or selected from the plurality of candidate beam geometries for which the increase in the cost function value is relatively higher, since the increase in the modified cost function value with these particular beam geometries removed compared to the original cost function value based on all candidate beam geometries indicates that the removed beam geometries are more optimal.

Finally, the chosen set B of beam geometries will contain those beam geometries which have optimal beam geometries with respect to the cost function.

In general, the method is equally applicable to IMPT for selection of suitable beam geometries.

Further, it is preferred that the method comprises providing treatment parameters and/or tolerance parameters for different regions of interest. For example, dose-volume costs and/or dose-volume constraints and/or importance factors for different regions of interest, in particular structures or organs at risk, may be provided as tolerance parameters. For example, prescribed radiation doses and/or minimal radiation doses and/or exposure time to radiation, in particular for volumes to be treated, may be provided as treatment parameters.

Preferably, treatment parameters and/or tolerance parameters for different regions of interest are provided in the beginning of the method, preferably before optimization or computation of cost function values takes place. The treatment parameters and/or tolerance parameters for different regions of interest can, for example, be defined by a user or provided as templates, for example as standard anatomical templates, which can be based, for example, on Radiation Therapy Oncology Group (RTOG) or institution-specific protocols. Those templates can be, for example, stored in a memory of a system or computer and selected to be provided for the method. In particular, different templates may be provided for different body sites, such as brain, head and neck, lung, prostate, et cetera.

The method as described herein has various advantages. Firstly, the method according to the invention combines the advantages of both exhaustive search algorithms and ranking algorithms by using the cost function for selecting a set of beam geometries B from a pool of candidate beam geometries P. Further, the method accounts for various synergistic or interplay effects between different beam geometries. Further, the method directly uses the cost function to choose beam geometries and hence the beam geometries resulting from the method described herein will be optimal with respect to clinical goals.

In a preferred embodiment, the method comprises optimizing the radiation treatment plan with a one or more chosen beam geometries. It is preferred that the selected set of beam geometries is used in a radiation treatment plan and the radiation treatment plan is optimized prior to the treatment based on the one or more chosen beam geometries.

In a further preferred embodiment, the optimization of the radiation treatment plan is applied at fluence level using fluence map optimization (FMO) and/or at control-point level using direct machine parameter optimization (DMPO). The optimization of the radiation treatment plan is applied, for example, at fluence level, preferably using fluence map optimization, and/or at control-point level, for example using direct machine parameter optimization. Fluence map optimization (FMO) is described, for example, in S. V. Spirou, C.-S. Chui. A gradient inverse planning algorithm with dose-volume constraints. Med Phys 1998; 25: 321-33 and Q. Wu, R. Mohan. Algorithm and functionality of an intensity modulated radiotherapy optimization system. Med Phys 2000; 27:701-11, which are incorporated herein by reference. Direct machine parameter optimization (DMPO) is described, for example, in B. Hardemark, A, Liander, H. Rehbinder and J. Löf, “Direct Machine Parameter Optimization with RayMachine in Pinnacle,” RaySearch White Paper, 2004, which is incorporated herein by reference. Preferably, the initial radiation treatment plan based on all candidate beam geometries as well as the final radiation treatment plan based on the one or more chosen beam geometries is optimized as previously described. In the context of direct machine parameter optimization, highly impactful control points can be sampled only for which beam geometry selection can be done.

According to a further preferred embodiment, the method comprises a dose computation for the one or more chosen beam geometries. Preferably, the dose computation for the one or more chosen beam geometries takes place after optimization of the final radiation treatment plan based on the one or more chosen beam geometries. Further preferably, a dose computation or calculation engine is applied in dose computation. For example, Pencil Beam Algorithm and/or Collapsed Cone Convolution (CCC) and/or Analytical Anisotropic Algorithm (AAA) may be used in dose computation.

Preferably, information like treatment and/or tolerance parameters, such as dose-volume costs and/or dose-volume constraints and/or importance factors, for different regions of interest are taken into consideration in dose computation.

Preferably, a final treatment plan is obtained by a final fluence map optimization and is followed by dose computation for the beams in the selected set B. Further, it is preferred that a final cost function value based on the set of selected or chosen beam geometries is computed.

Unlike exhaustive search techniques, the method described herein can be used with just two cycles of optimization to select suitable beam geometries and hence is very fast compared to exhaustive search techniques.

In a further preferred embodiment, the plurality of candidate beam geometries comprises beam or gantry angles and/or couch angles and/or collimator angles. In particular, a beam geometry may refer to a beam angle or gantry angle (in case of coplanar beams) and/or couch angles (in case of non-coplanar beams) and/or collimator angles. Since each beam geometry may comprise these angles, the plurality of candidate beam geometries also comprises information about these angles.

According to a further preferred embodiment, the plurality of candidate beam geometries comprises several equiangular beam geometries or is biased. Typically, the pool of candidate beams will contain several equiangular beams. However, in some situations, it could be useful to bias the plurality of candidate beams upfront to favor a particular range of angles. For example, the biasing of the plurality of candidate beams can be employed to fetch more clinically relevant beam geometries in some complicated body sites such as lung cases.

In a further preferred embodiment, the plurality of candidate beam geometries is based on a beam geometry template. The beam geometry template preferably comprises a beam angle template. For example, beam geometry or beam angle templates have been published for different body sites, such as brain, head and neck, lung, prostate, et cetera. Beam geometry templates preferably can be used to create a plurality of candidate beam angles most appropriate for the treatment situation.

In another embodiment it is preferred that choosing one or more beam geometries is conducted using an algorithm taking into account the modified cost function values and further, preferably, a total number of chosen beam geometries and/or a minimum angle gap between two successive beam geometries. As an alternative to choosing the one or more beam geometries by a user, for example, this step preferably is conducted automatically, in particular fully automated, by using an algorithm. This algorithm preferably picks an optimal set of beam geometries by taking into account the modified cost function values. In particular, it is preferred that the algorithm is configured to choose those beam geometries, for which the modified cost function value has a relatively high value. Further, it is preferred that the algorithm is based on the total number of chosen beam geometries, i.e. the total number of beam geometries that are to be chosen with the method. Depending on this total number of beam geometries to be chosen, the algorithm preferably picks those beam geometries which are particularly useful according to the modified cost function values. Preferably also a minimum angle gap between two successive beam geometries is considered. This minimum angle gap can also be referred to as minimum beam angle constraint.

In a further preferred embodiment, as a modified cost function value, the square of the resulting cost function value is used, i.e. the square of the direct calculated result from the computation of the cost function for the candidate beam geometries without the removed beam geometry as the modified cost function value. The square can be used in order to perceive the differences in the modified cost function values in a better way.

Further, it is preferred that the modified cost function values are plotted over the different beam geometries, in particular the different beam angles. Further preferably, the one or more beam geometries are chosen, preferably by an algorithm as described above, from such a plot of the modified cost function values.

In a further aspect of the invention, a system for selecting a set of beam geometries for use in radiation therapy is presented, wherein the system comprises: a processor in communication with a memory, where the memory stores program code and the processor is configured to be operative in conjunction with the program code to:
provide a plurality of candidate beam geometries;
optimize a radiation treatment plan with all candidate beam geometries;
compute a cost function value based on all candidate beam geometries;
remove a first beam geometry from the plurality of candidate beam geometries;
compute a first modified cost function value based on the candidate beam geometries without the removed first beam geometry;
restore the first beam geometry to the plurality of candidate beam geometries;
repeat the steps of removing a beam geometry, computing of a modified cost function value and restoring of the removed beam geometry for all other candidate beam geometries;
choose one or more beam geometries from the plurality of candidate beam geometries based on the modified cost function values.

In a another aspect of the invention, a computer program for selecting a set of beam geometries for use in radiation therapy is presented, wherein the computer program comprising program code means for causing a system for selecting a set of beam geometries for use in radiation therapy as previously defined to carry out the steps of the method as defined in claim 1, when the computer program is run on a computer controlling the system for selecting a set of beam geometries for use in radiation therapy.

It shall be understood that the method of selecting a set of beam geometries for use in radiation therapy of claim 1, the system for selecting a set of beam geometries for use in radiation therapy claim 10, and the computer program for selecting a set of beam geometries for use in radiation therapy of claim 11 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a cross-sectional plan view of an embodiment of an arrangement with a couch and a radiation source;

FIG. 2 shows schematically and exemplarily an enlarged isometric view of a treatment beam source for use with the arrangement shown in FIG. 1;

FIG. 3 shows schematically and exemplarily a flowchart representing steps of an embodiment of a method of selecting a set of beam geometries for use in radiation therapy;

FIG. 4 shows schematically and exemplarily an embodiment of a system for selecting a set of beam geometries for use in radiation therapy;

FIG. 5 shows schematically and exemplarily a plot of modified cost function values for different beam angles;

FIG. 6 shows schematically and exemplarily a dose-volume histogram comparison;

FIG. 7 shows schematically and exemplarily two sets of beam geometries.

### DETAILED DESCRIPTION OF EMBODIMENTS

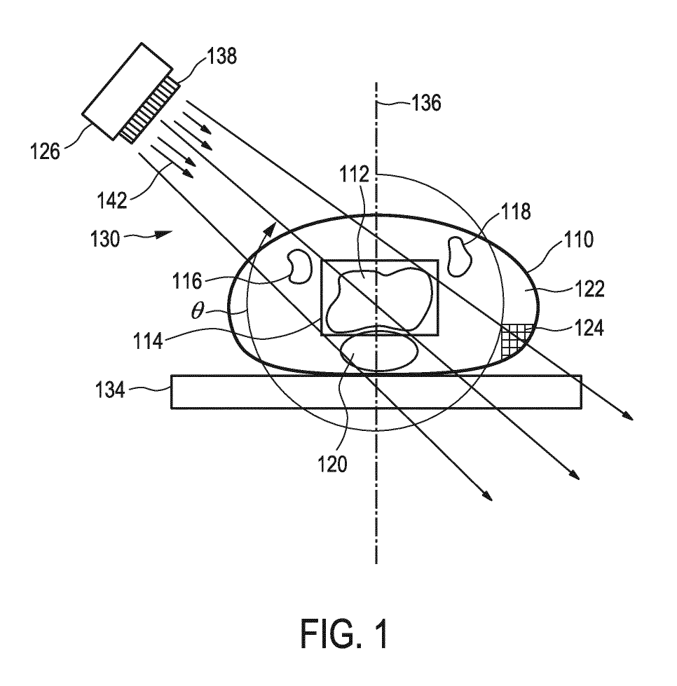

FIG. 1 schematically and exemplarily shows an embodiment of an arrangement with a model 110 of a patient geometry, which is used for treatment planning, for example in an intensity modulated radiation therapy (IMRT) before performing any therapy on the patient. Model 110 may be obtained by any suitable technique, for example computed tomography (CT) or magnetic resonance imaging (MRI) scans of the body can be used to obtain model 110. For the purposes of the therapy, a structure to be treated by radiation, in this case a tumor 112, is identified and localized within a planning target volume (PTV) 114. Further, structures at risk such as organs 116, 118, bone 120 and tissue 122 are identified.

For treatment planning, patient model 110 may be divided into a three-dimensional grid defining a number of voxels 124, of which only a few are indicated in FIG. 1 for reasons of clarity. All voxels 124 within PTV 114 are assigned a treatment parameter value necessary to treat tumor 112. The treatment parameter can be expressed in term of a radiation dose which is to be delivered to tumor 112, exposure time to radiation and/or various other parameters. For example, the treatment parameter can be chosen to be a radiation dose prescribed for treating tumor 112. This radiation dose is to be delivered to each voxel 124 in PTV 114. In table I further described below, the treatment parameter is given as a minimal or uniform dose in the form of target radiation or target dose in cGy, for example.

A tolerance parameter may be assigned to structures at risk 116, 118, 120, 122. As in the case of the treatment parameter, the tolerance parameter can be expressed in terms of dose and/or other parameters. For example, the tolerance parameter may be chosen to be a tolerance dose. In table I described below, for different structures at risk the tolerance parameter is given as a maximum dose or maximum dose volume histogram in the form of target radiation or target doses cGy for the maximum dose and in addition with a % volume for the maximum dose volume histogram.

FIG. 1 shows a radiation source 126 such as a linear accelerator for providing a treatment beam 130. The orientation of the treatment beam 130 is typically determined in terms of an angle of a couch 134 on which the patient is positioned, commonly referred to as the couch angle, and further in terms of a beam or gantry angle θ at which source 126 is inclined with respect to the normal 136. Both angles can be varied in discrete increments and in principle continuously over predetermined ranges. In the present description, only adjustments in the beam or gantry angle θ over the range of 0 degrees to 360 degrees are shown, but it is to be understood that the couch angle can be adjusted as well. For example, the International Electronical Commission (IEC) convention can be used in defining and adjusting both gantry and couch angles. In case of coplanar beam treatment, the couch angle can be set to 0 degrees, otherwise the couch angle adjustments can vary, for example, in increments of several degrees (e.g., 10 degrees) from −20 degrees to 20 degrees (e.g., for paraspinal patient treatment) or from −40 degrees to 40 degrees (e.g., for a head-and-neck treatment) for non coplanar beam treatments.

Radiation source 126 preferably is equipped with beam guiding and shaping mechanism 138 for subdividing treatment beam 130 into a number of component beams or beamlets 142. Mechanism 138 generally includes a leaf collimator, in particular a multi-leaf collimator (MLC), for collimating beamlets 142 as well as additional lensing devices, apertures, masks and other elements suitable for guiding and shaping of beamlets 142.

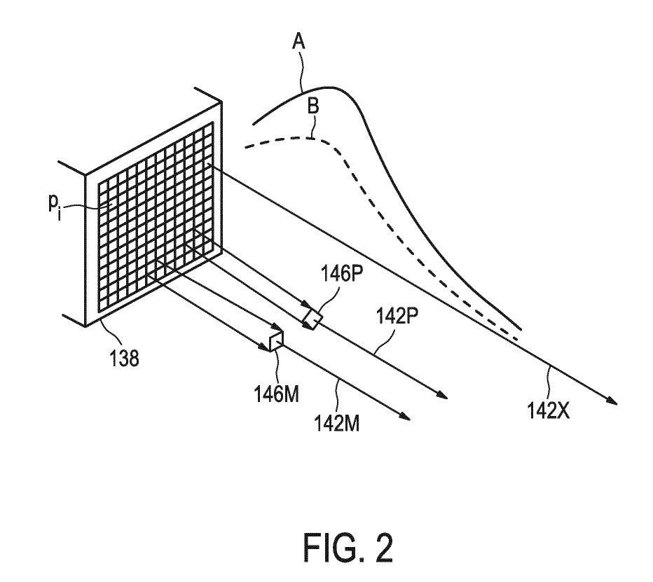

FIG. 2 illustrates mechanism 138 of radiation source 126 generating treatment beam 130 from a number of beamlets 142 with the aid of mechanism 138 in more detail. Mechanism 138 is drawn to indicate “pixels” pᵢ corresponding to each beamlet 142. The number of beamlets 142 in beam 130 will depend on the cross-section of treatment beam 130 necessary for irradiating PTV 114 at the required resolution, for example. Typically, the number of beamlets 142 ranges from a few tens to a few hundred or more.

Radiation source 126 has the capacity of modulating various parameters of beamlets 142. For example, mechanism 138 of source 126 can be used to modulate the cross-section of beamlets 142. This is shown on the examples of particular beamlets 142M and 142P. Cross-sections 146M, 146P of beamlets 142M and 142P are square or rectangular. Cross-section 146P is rotated around the direction of propagation of beamlet 142P by 90 degrees. The rotation can be performed by rotating individual collimators of mechanism 138 corresponding to beamlets 142M, 142P. It can be preferred to rotate the cross-sections of all beamlets together.

Radiation source 126 may also be capable of modulating the amount of energy in the beamlets. For example, beamlet 142X shows radiation doses A and B as function of distance from source 126 produced at high energy and at low energy. The same collimation and focusing parameters are used at both beamlet energies. The energy levels drop off exponentially with distance and so the doses experience exponential attenuation. Preferably, source 126 can also modulate the on and off times of beamlets 142.

All of the above beamlet parameters can be adjusted independently or in combination by radiation source 126 and mechanism 138 in attempting to deliver the prescribed radiation dose to PTV 114. It is understood that additional radiation sources equivalent to source 126 can be employed.

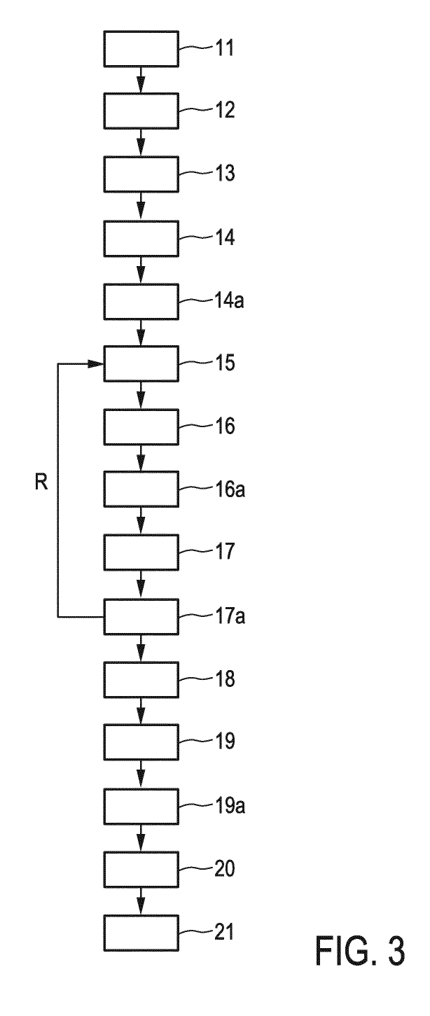

FIG. 3 schematically and exemplarily shows process steps of an embodiment of a method 10 of selecting a set of beam geometries for use in radiation therapy, for example in an arrangement shown in FIGS. 1 and 2.

In a first step 11, preferably treatment parameters and/or tolerance parameters, such as dose-volume costs and/or dose-volume constraints and/or importance factors and/or radiation doses for different regions of interest can be provided. In particular, tolerance parameters may be provided based on standard anatomical templates (e.g., Radiation Therapy Oncology Group or institution-specific protocols).

In a subsequent method step 12, it is preferred to provide a plurality of candidate beam geometries P. The plurality of candidate beam geometries can comprise several equiangular beam geometries or can be biased. The plurality of candidate beam geometries can also be based on a beam geometry template for a specific body site, for example.

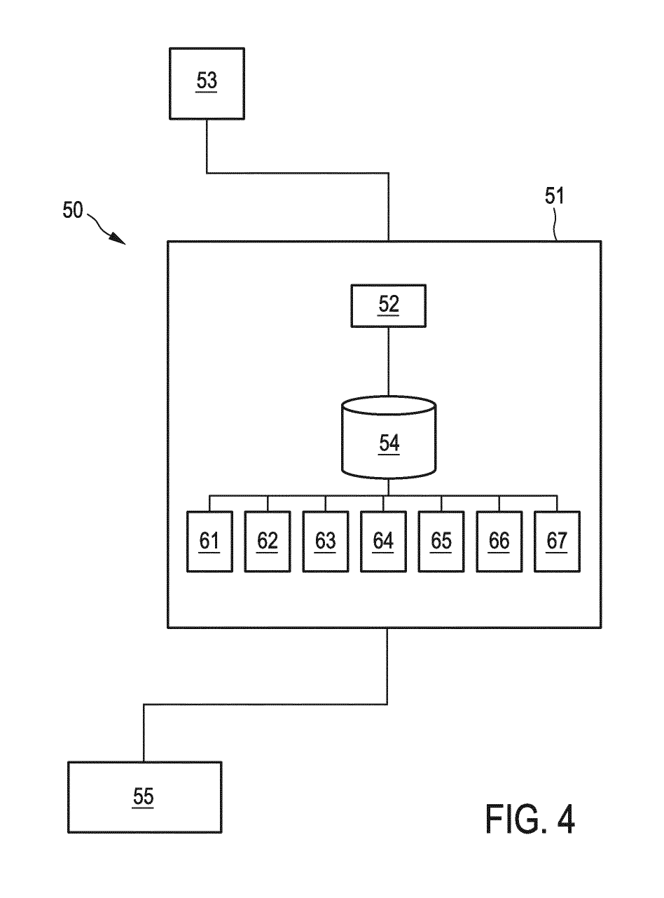

The provision of the treatment parameters and/or tolerance parameters as well as the provision of the plurality of candidate beam geometries may be conducted based on a user's input, preferably via a user interface 53 of a system 50 as shown in FIG. 4 and/or provided from information stored in memory 54 of a system 50 shown in FIG. 4.

In the next step 13, a radiation treatment plan is optimized with all candidate beam geometries in pool P. Subsequently, in step 14, the respective composite cost function value F is computed. This original or composite cost function value is then preferably stored in step 14a. Steps 11 to 14a can be summarized as initial optimization and dose computation.

After this initial optimization and dose computation, a first beam geometry is removed from the plurality of candidate beam geometries in step 15. For example, a dose contribution from the first beam geometry can be removed by assigning a beam weight value of zero. This can be done, for example, in a treatment planning system by switching off a beam's monitor unit (MU), preferably without the need to invalidate the computed dose or optimized fluence map.

In the following step 16, a modified cost function value Fₘ based on the candidate beam geometries without the removed first beam geometry is computed. The modified cost function value Fₘ can be the square of the direct calculated result cost function or the respective change in the cost function value. This first modified cost function value Fₘ is then stored in step 16a.

These steps 15 through 16a, can be referred to as beam reduction.

Subsequently, the previously removed first beam geometry is restored in method step 17. In particular, the dose contribution from the removed first beam geometry is restored, preferably by resetting its beam weight to the original optimized value. It is particularly preferred that this can be done in a treatment planning system by switching on a beam's monitor unit (MU).

In a further subsequent step 17a, the original or composite cost function value F is restored back. These steps 17, 17a can be summarized as retaining the beam contribution.

In the following, the steps 15 through 17a are repeated for all remaining candidate beam geometries in the pool P, as indicated with arrow R in method 10 in FIG. 3.

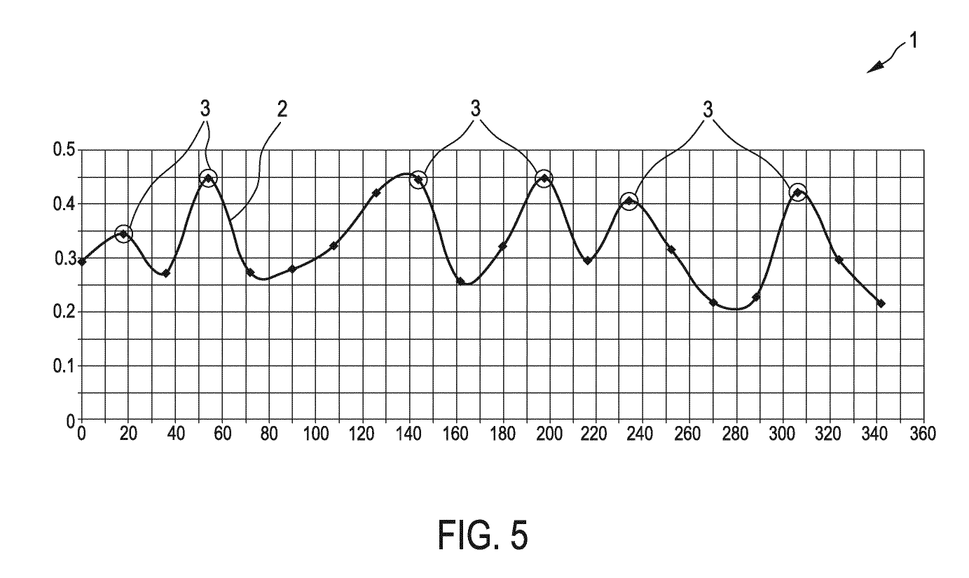

In a further step 18, the modified cost function Fₘ corresponding to the removal of each beam from the pool of candidate beams is plotted as a graph over the different beam geometries, in particular the different beam angles, as shown schematically and exemplarily in FIG. 5. FIG. 5 shows a plot 1 of the modified cost function values 2 as the square of the direct calculated result of the cost function for the different beam angles between 0 and 360 degrees. Line 2 indicates the modified cost function values with respect to removal of each beam geometry in the pool of candidate beam geometries.

In subsequent step 19, one or more beam geometries from the plurality of candidate beam geometries are chosen based on the modified cost function values 2 shown in FIG. 5. In particular, the modified cost function values 3 which belong to the selected beam geometries forming the set of selected beam geometries B for use in the radiation therapy are indicated in FIG. 5. In addition to considering the modified cost function values, a total number of beams to be selected and a minimum angle gap preferably are also considered when one or more beam geometries are chosen.

The one or more chosen beam geometries can be transferred to a set of selected beam geometries B in a step 19a. Preferably, steps 19 and 19a are fully automated by using an algorithm as described above. Steps 19 and 19a are also referred to as beam angle selection.

In a following process step 20, a final treatment plan optimization based on the one or more selected beam geometries is conducted, followed by a dose computation or dose calculation 21. Steps 20 and 21 can also be referred to as final optimization and dose computation.

FIG. 4 schematically and exemplarily shows an embodiment of a system 50 for selecting a set of beam geometries for use in radiation therapy, which can be used preferably with the method 10 shown in FIG. 3. The system 50 comprises a processor or central processing unit 52 in communication with a memory 54. The processor 52 and memory 54 may be part of a computer 51. The computer preferably is connected to a user interface 53 to receive input from a user and/or to provide an output to a user. Further preferably, the computer 51 is connected to a treatment device 55, which preferably comprises a radiation source and potentially an arrangement as shown in FIGS. 1 and 2. The memory 54 preferably stores program code 67 and the processor 52 preferably is configured to operate in conjunction with the program code 67 to execute the steps of method 10. The program code preferably is processor-executable.

Additional information which can preferably be stored in memory 54 may comprise the pool P of candidate beams 61, beam angle templates 62, the composite or original cost function value F 63, the modified cost function values Fₘ 63, the set B of selected beam geometries 65 and/or treatment and/or tolerance parameters 66. It is to be noted that not all of this information needs to be stored in memory 54 and that further additional information can also be stored in memory 54.

The modified cost function values 2 shown in plot 1 shown in FIG. 5 have been obtained for a second set of beam geometries in an example case for treatment of the head-and-neck region. For comparison, two sets of beam geometries were defined: A first set of beam geometries was defined using equiangular logic. The second set of beam geometries was defined using the method described herein. The treatment and tolerance parameter specified for different regions of interest (ROIs) according to the following table I were kept the same for both sets of beam geometries.

TABLE I
Important dose-volume costs specified

| | | Target | % | % | |
|---|---|---|---|---|---|
| ROI | Type | cGy | Volume | Variation | Weight |
| Cord | Max Dose | 4200 | | | 25 |
| Parotis RT | Max DVH | 4700 | 50 | | 4 |
| Parotis LT | Max DVH | 3300 | 50 | | 4 |
| Subman RT | Max DVH | 6300 | 50 | | 4 |
| Subman LT | Max DVH | 4800 | 50 | | 1 |
| PTV50imrt | Min Dose | 4750 | | | 30 |
| PTV60imrt | Min Dose | 5700 | | | 50 |
| PTV66imrt | Uniform Dose | 6630 | | | 1 |
| PTV66imrt | Min Dose | 6270 | | | 25 |
| Spinal Cord_PRV | Max DVH | 4600 | 0 | | 25 |

Also, the initial fluence map optimization was conducted in the same way for both sets of beam geometries. Conversion was not included. Conversion is a process of converting a given fluence map into a set of deliverable multi-leaf collimator (MLC) segment. A segment is a particular spatial configuration of MLC leafs. This will happen for each beam independently. Some additional important parameter settings used are: beam angle resolution=18 degrees, minimum beam angle constraint=40 degrees, CT slice thickness=0.3 cm, dose calculation grid size=0.3 cm in all directions. The comparison of the final treatment plans derived from the beam angle geometries of the first set and the second set was done in terms of dose distribution (conformity), dose-volume histogram, final cost function value and total monitor units (MUs). In the following table II, the results for the first set of beam geometries without a beam angle optimization according to the method described herein is compared to the second set of beam geometries with a beam angle optimization according to the method described herein.

TABLE II
Comparison of Cost Function Value and Total Monitor Unit

| | Without BAO | With BAO |
|---|---|---|
| Final Cost Function Value | 0.6112 | 0.5815 |
| Total Monitor Unit (MU) | 1212 | 1074 |
| Conformity Index | 1.324 | 1.317 |

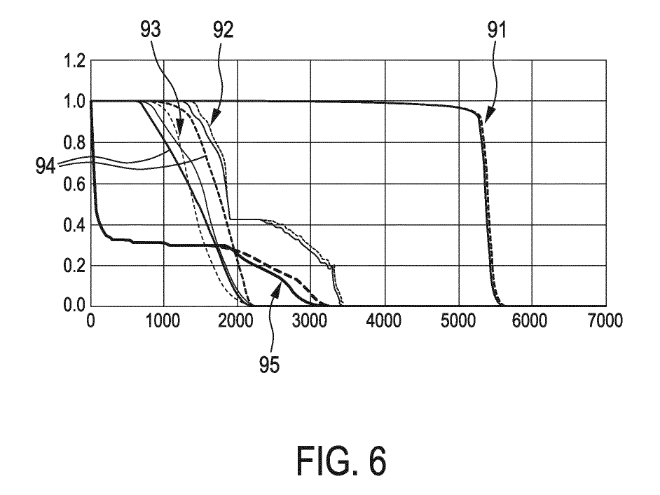

Further, the dose-volume histogram shown in FIG. 6 denotes on the horizontal axis the dose (cGy (relative biological effectiveness (RBE)). The continuous lines show the dose for the second set of beam geometries selected according to the method described herein and the dotted lines show the dose for the set of equiangular beams without selection according to the method described herein for different regions of interest, namely lines 91 for the patient treatment volumes PTV 54-72, lines 92 for the chiasm, lines 93 for the left parotid, lines 94 for the right parotid and lines 95 for the cord.

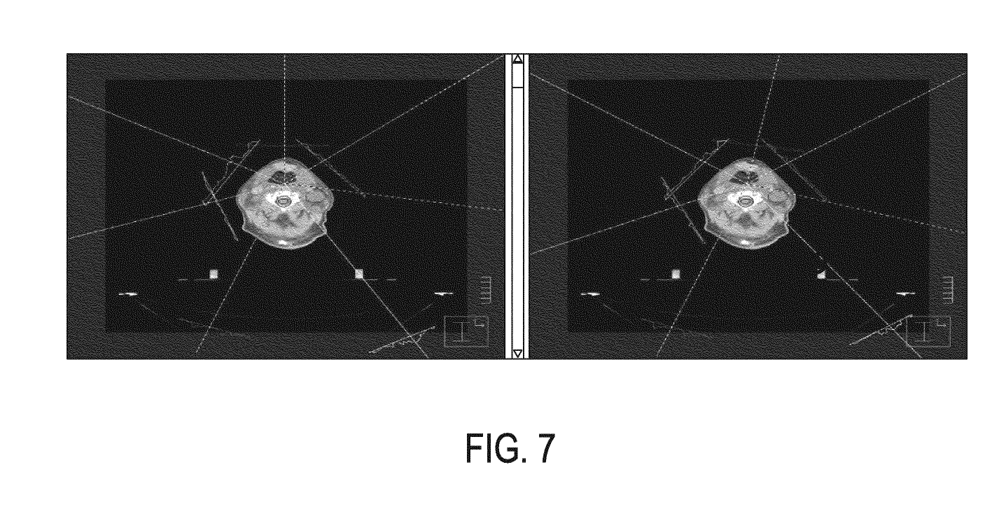

FIG. 7 indicates the beam angles for the first set of equiangular beam geometries on the left side and the beam angles chosen according to the method described herein for the second set on the right side.

The method, system and computer program for selecting a set of beam geometries for use in radiation therapy, in particular IMRT, described herein directly uses the cost function to choose beam geometries and accounts for various synergistic effects between beams. The resulting beams of the method described herein therefore are optimized with respect to clinical goals, while at the same time, the method described herein is much faster than exhaustive search techniques.

The goal of radiation therapy is to deliver a prescribed dose of radiation usually in the form of electromagnetic radiation (photons), electrons, neutrons or protons to a treatment target or target volume, such as a tumor, while sparing adjacent structures or organs at risk (OARs). The treatment target or target volume and structures or organs at risk are also referred to as regions of interest (ROIs). In intensity modulated radiation therapy (IMRT) the intensity profiles of the incident beams are modulated to achieve a better dose distribution.

Radiation sources emitting the beams can be arranged on a frame or gantry, which usually is rotatable about an axis of rotation, wherein this axis of rotation may be identical to or parallel to a longitudinal axis of a couch or table, on which a patient can be placed. Sources for emitting the radiation beams are usually equipped with beam guiding and shaping mechanisms for subdividing treatment beams into a member of component beams or beamlets. Those guiding and shaping mechanisms may include leaf collimators, in particular multi-leaf collimators (MLC), for collimating beamlets as well as additional lensing devices, apertures, masks and other elements suitable for guiding and shaping of beamlets. For example, a guiding and shaping mechanism of a radiation source may be used to modulate the cross-section of beamlets. For example, beamlets with square or rectangular cross-sections may be formed. Further, the cross-section of the beamlet may be rotated around the direction of propagation of a beamlet, for example by 90 degrees. Such a rotation can be performed by rotating individual collimators of the guiding and shaping mechanism corresponding to the beamlets to be rotated. The degree to which the cross-section or the beamlet is rotated can be referred to as collimator rotation and can be indicated by the collimator angle.

In treatment planning the beam geometries at which radiation is delivered to the treatment site in the patient's body, are usually pre-selected based on experience and intuition of the operator. A beam geometry usually comprises the beam angle, also called gantry angle, for a coplanar beams. For non-coplanar beams the beam geometries can also comprise couch angles. Further, collimator angles can be comprised. Each beam usually is subdivided into a number of component beams or beamlets.

The corresponding beam intensity profiles are then optimized under the guidance of an objective function, typically using so-called inverse treatment planning methods.

However, existing approaches to beam orientation selection in radiation therapy have a number of disadvantages. Computation time required by a complete beam orientation optimization using exhaustive search algorithms is prohibitively long, and hence not suitable for clinical applications. Ranking or scoring algorithms are fast but not accurate as the intent is to solve the beam angle selection problem independent of the problem of treatment plan optimization, usually a fluence map or segment optimization. In addition, mostly the cost function used to address the beam angle selection problem is different from the cost function used for the optimization problem of the treatment plan. Using a different ranking function may result in a beam orientation that is not optimal with respect to the cost function used for final treatment plan optimization. Moreover, the majority of the ranking algorithms do not represent potential synergetic effects of beam combinations and thus do not account for the beam interplay effect, which leads to an inaccurate selection of beam angles. Trial and error attempts are often needed in order to determine a set of good beam geometries for radiation treatment.

The selection of optimal beam geometry has been of interest since the advent of 3D conformal radiation therapy. Currently, the beam angle selection process in IMRT is based on the experience of the treatment planners or by a trial-and-error approach. It has been attempted to automate the beam placement process in IMRT. In general, the optimization of the beam angles may have a significant influence on the quality of an IMRT treatment. It has been demonstrated that the plans with fewer but optimized beam angles could be equal to or even superior to the plans with a larger number of unoptimized beam angles, as described in V. K. Narayanan, R. Vaitheeswaran, J. R. Bhangle, S. Basu, V. Maiya, and B. Zade, “An experimental investigation on the effect of beam angle optimization on the reduction of beam numbers in IMRT of head and neck tumors,” J. Appl. Clin. Med. Phys. 13, 36-43 (2012), which is incorporated herein by reference. A fewer number of beams in a plan generally leads to shorter treatment time and hence lower probability of patient movement related errors during treatment delivery. However, the planner's intuition about the beam angles that generally works for 3DCRT may not work well for an IMRT situation mainly due to the fact that the beam directions are inseparably coupled with the intensity maps of the incident beams. Hence, several trial-and-error attempts are usually needed to find out a set of acceptable beam angles in IMRT.

Among optimization algorithms employed for solving beam angle optimization (BAO) problem, are genetic algorithms (GA), as described in J. Lei and Y. J. Li, “An approaching genetic algorithm for automatic beam angle selection in IMRT planning,” Comput. Methods Programs Biomed. 93, 257-265 (2009), which is incorporated herein by reference, and Simulated Annealing (SA) algorithms, as described in D. Djajaputra, Q. W. Wu, Y. Wu, and R. Mohan, “Algorithm and performance of a clinical IMRT beam-angle optimization system,” Phys. Med. Biol. 48, 3191-3212 (2003), which is incorporated herein by reference. Such exhaustive search algorithms generally explore a large number of candidate solutions to arrive at an optimal beam configuration, which considerably prolongs the entire process of BAO in IMRT. Although it is feasible to use GA and SA to solve the BAO problem, the convergence speed from the computational perspective is not satisfying, especially for routine clinical use, as described in P. S. Potrebko, B. M. C. McCurdy, J. B. Butler, A. S. El-Gubtan, and Z. Nugent, “A simple geometric algorithm to predict optimal starting gantry angles using equiangular-spaced beams for intensity modulated radiation therapy of prostate cancer,” Med. Phys. 34, 3951-3961 (2007), which is incorporated herein by reference.

Different beam angle ranking techniques for selection of beam angles in IMRT are described in M. Braunstein and R. Y. Levine, “Optimum beam configurations in tomographic intensity modulated radiation therapy,” Phys. Med. Biol. 45, 305-328 (2000); Gaede, H. Rasmussen, and E. Wong, “An algorithm for systematic selection of beam directions for IMRT,” Med. Phys. 31, 376-388 (2004); or P. S. Potrebko, B. M. C. McCurdy, J. B. Butler, A. S. El-Gubtan, and Z. Nugent, “A simple geometric algorithm to predict optimal starting gantry angles using equiangular-spaced beams for intensity modulated radiation therapy of prostate cancer,” Med. Phys. 34, 3951-3961 (2007), which are all incorporated herein by reference. These methods use some metric to evaluate the value of a given beam direction. The metric is evaluated for each beam of a set of candidate orientations and the top ranking orientations are used for a subsequent optimization. Such ranking techniques are faster than the other exhaustive search approaches. However, such ranking techniques ignore the interplay effects between the beams. A ranking based method has recently been described that incorporates beam interaction in the selection process described in R. Vaitheeswaran, V. K. Narayanan, J. R. Bhangle, A. Nirhali, N. Kumar, S. Basu, and V. Maiya, “An algorithm for fast beam angle selection in intensity modulated radiotherapy,” Med. Phys. 37, 6443-6452 (2010), which is incorporated herein by reference. However, the ranking function is different from the cost function used for the final plan optimization. Using a different ranking function may result in a beam orientation that is not optimal with respect to the cost function used for final plan optimization, as described in Popple, Richard A., Ivan A. Brezovich, and John B. Fiveash. “Beam geometry selection using sequential beam addition,” Med. Phys. 41.5 (2014), 051713, which is incorporated herein by reference. Further methods have been described in U.S. Pat. No. 7,876,882 B2, U.S. Pat. No. 6,504,899 B2 and US 2012/0136194 A1, which are all incorporated herein by reference. A hybrid approach to beam angle optimization in IMRT has been described in Bertsimas D, et al. A hybrid approach to beam angle optimization in intensity-modulated radiation therapy. Computers and Operations Research (2012), http://dx.doi.Org/10.1016/j.cor.2012.06.009, which is incorporated herein by reference.

The method, system and computer program and their embodiments described herein provide an improved solution for fast and accurate beam selection with improved optimization results with respect to clinical goals.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

The invention relates to a method of selecting a set of beam geometries for use in radiation therapy. The method comprises providing a plurality of candidate beam geometries; optimizing a radiation treatment plan with all candidate beam geometries; and computing a cost function value based on all candidate beam geometries. A first beam geometry from the plurality of candidate beam geometries is removed and a first modified cost function value based on the candidate beam geometries without the removed first beam geometry computed. The first beam geometry is restored. The steps of removing a beam geometry, computing of a modified cost function value and restoring of the removed beam geometry are repeated for all other candidate beam geometries. One or more beam geometries from the plurality of candidate beam geometries based on the modified cost function values are choosen.

1. A method of selecting a set of beam geometries for use in radiation therapy, the method comprising,
providing a plurality of candidate beam geometries;
optimizing a radiation treatment plan with all candidate beam geometries;
computing a cost function value based on all candidate beam geometries;
removing a first beam geometry from the plurality of candidate beam geometries;
computing a first modified cost function value based on the candidate beam geometries without the removed first beam geometry;
restoring the first beam geometry to the plurality of candidate beam geometries;
repeating the steps of removing a beam geometry, computing of a modified cost function value and restoring of the removed beam geometry for all other candidate beam geometries;
choosing one or more beam geometries from the plurality of candidate beam geometries based on the modified cost function values.

2. The method according to claim 1,
comprising optimizing the radiation treatment plan with the one or more chosen beam geometries.

3. The method according to claim 1,
wherein the optimization of the radiation treatment plan is applied at fluence level using Fluence Map Optimization and/or at control-point level using Direct Machine Parameter Optimization.

4. The method according to claim 1,
comprising a dose computation for the one or more chosen beam geometries.

5. The method according to claim 1,
wherein the plurality of candidate beam geometries (P) comprises beam angles and/or couch angles and/or collimator angles.

6. The method according to claim 1,
wherein the plurality of candidate beam geometries (P) comprises several equiangular beam geometries or is biased.

7. The method according to claim 1,
wherein the plurality of candidate beam geometries (P) is based on a beam geometry template.

8. The method according to claim 1,
wherein choosing one or more beam geometries is conducted using an algorithm taking into account the modified cost function values, and/or a total number of chosen beam geometries, and/or a minimum angle gap between two successive beam geometries.

9. The method according to claim 1,
wherein as a modified cost function value (Fₘ), the square of the resulting cost function value is used.

10. A system for selecting a set of beam geometries for use in radiation therapy, the system comprising a processor in communication with a memory, where the memory stores program code and the processor is configured to be operative in conjunction with the program code to:
provide a plurality of candidate beam geometries;
optimize a radiation treatment plan with all candidate beam geometries;
compute a cost function value based on all candidate beam geometries;
remove a first beam geometry from the plurality of candidate beam geometries;
compute a first modified cost function value based on the candidate beam geometries without the removed first beam geometry;
restore the first beam geometry to the plurality of candidate beam geometries;
repeat the steps of removing a beam geometry, computing of a modified cost function value and restoring of the removed beam geometry for all other candidate beam geometries;
choose one or more beam geometries from the plurality of candidate beam geometries based on the modified cost function values.

11. A computer program for selecting a set of beam geometries for use in radiation therapy, the computer program comprising program code means for causing a system for selecting a set of beam geometries for use in radiation therapy as defined in claim 10 to carry out the steps of the method as defined in claim 1, when the computer program is run on a computer controlling the system for selecting a set of beam geometries for use in radiation therapy.
